(19) United States
(12) Patent Application Publication
(10) Pub. No.: US 2016/0089431 A1
(43) Pub. Date: Mar. 31, 2016

(54) Nanodisk-associated immunogen super polyvalent vaccines

(71) Applicants: Anthony David Bressi, Elysburg, PA (US); Dennis Szymanski, Ft. Dix, NJ (US)

(72) Inventors: Anthony David Bressi, Elysburg, PA (US); Dennis Szymanski, Ft. Dix, NJ (US)

(73) Assignee: SHIVA SCIENCE & TECHNOLOGY GROUP LLC, Elysburg, PA (US)

(21) Appl. No.: 14/497,751

(22) Filed: Sep. 26, 2014

(51) Int. Cl. A61K 39/21; C07K 17/04; G01N 33/544; A61K 39/145; C12N 7/00

(52) U.S. Cl. CPC A61K 39/21; A61K 39/145; C12N 7/00; G01N 33/544; C07K 17/04; C12N 2760/16134; C12N 2740/16034; A61K 2039/6018

(57) ABSTRACT

Disclosed are compositions of matter consisting of collections of nanodisks associated with polypeptide immunogens. Each said collection contains multiple epitopes of at least one immunogen. When used as a component of a vaccine, these epitopes are chosen to confer immunity, in the vaccinated subject, against a spectrum of possible infective strains of the target pathogen. This immunity is derived from the various antibodies, to the various epitopes of the immunogen, produced by the adaptive immune system of the subject in response to exposure to said vaccine. In this manner, said vaccine may confer immunity against the entire spectrum of possible infective strains of the target pathogen.

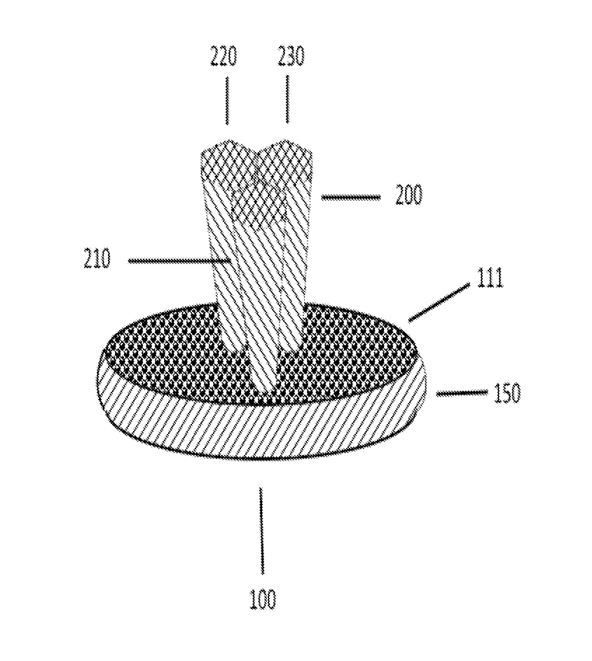

### CROSS-REFERENCE TO RELATED APPLICATIONS

This application claims the priority benefit under 35 USC 119(e) from Provisional Application No. U.S. 61/883,101, “Nanodisk-associated immunogen super polyvalent vaccines,” filed September, 2013 and which is incorporated herein in its entirety.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### FIELD OF THE INVENTION

The present invention relates to immunology; immunologically-based assays, detection, screening, isolation, extraction, and purification technology; and the production of vaccines which protect against a variety of pathogens.

### BACKGROUND OF THE INVENTION

Vaccination is widely accepted as the preferred, and, generally, most effective, method of preventing infection by a variety of pathogens. Conventional vaccines against viral pathogens usually consist of inactivated virus particles or live attenuated viruses. These are cultured in live cell systems, such as embryonated chicken eggs in the case of influenza A vaccines. As a result, the vaccine may contain residual antigens from these live cell systems. This poses a problem for persons with a hypersensitivity to these antigens. Vaccines against bacteria and similar microbes usually consist of dead, sometimes lysed, microbes. Sometimes the microbe is a different strain of, or a species related to, the pathogen. But as with the viral vaccines, the goal of the vaccine is to confer immunity, in the vaccinated subject, against the target pathogen.

This is accomplished if the adaptive immune system of the vaccinated subject generates sufficient antibodies capable of binding with the immunogens presented by the pathogen. These antibodies, when bound to the target immunogens, may block the ability of the pathogen to bind to, or enter, the cells of the subject. Another way in which these antibodies may confer immunity is by priming, or marking, the pathogen for destruction by other parts of the immune system.

More recently, attention has turned to the development of “subunit” vaccines. Instead of the whole, or lysed, pathogen, a subunit vaccine is composed of the actual polypeptide immunogen, or immunogens. Some of the potential advantages of such vaccines, produced using recombinant DNA techniques, would include ease of production, short pre-production time, lower production costs, ease of producing polyvalent vaccines, absence of safety concerns deriving from hypersensitivity to residual non-human animal antigens deriving from the culture system, absence of safety concerns deriving from exposing individuals with weak or compromised immune systems to a live, if weakened, virus that could produce infection in these individuals, and fairly rapid production cycles. However, subunit vaccines have, generally, proven to be disappointing. Even though these vaccines elicit high antibody titers in the vaccinated subject, these antibodies seem to have poor affinity for the immunogen presented by the target pathogen.

This poor affinity for the immunogen as it appears on the pathogen seems to stem from a conformation problem. The structure of most immunogens when not embedded in a viral envelope or a cellular membrane is different from when said immunogens are dissolved in an aqueous environment. This structural, or conformational, difference appears to be the cause of the discrepancy of poor activity against the target pathogen despite high antibody titers.

Virus-like particles, which are essentially the viral capsid either wrapped around an inert core, often simply a liposome, offer a way of presenting the immunogens to the adaptive immune system in the correct conformation to confer immunity against the target pathogen. However, these particles are difficult to produce in bulk quantities. Nanodisks are also capable of providing the proper environment for the polypeptide immunogen to exist in the necessary conformation to elicit efficacious immunogenicity.

Nanodisks are self-assembling and consist of a hydrophobic membrane core of lipids surrounded by a scaffold composed of two molecules of an amphipathic protein, called a membrane scaffolding protein (“MSP”), which encircle the lipid membrane. This lipid membrane may host polypeptides, including many immunogens, and may provide a suitable environment resembling that of a viral envelope or cell membrane. Recombinant hemagglutinin (“rHA”) from a single strain of influenza A virus has been successfully incorporated into nanodiscs by P. Bhattacharya et al in 2009 and the resulting formulation demonstrated efficacy comparable to a live-attenuated virus vaccine. This formulation also demonstrate some, though differing, levels of efficacy against other strains of the influenza A virus.

Several pathogens, including human immunodeficiency virus (“HIV”), have proven difficult to successfully vaccinate against because they present highly variable immunogens or occur in a great many strains and thus present a great many epitopes of the immunogens, such as influenza A virus. One way to generate the large variety of antibodies needed to neutralize or act against this great variety of immunogen epitopes, and thus create an effective vaccine, is for the vaccine to consist of a large collection of said immunogen epitopes. Such a super polyvalent vaccine would be difficult and expensive to produce using conventional vaccine production techniques.

### BRIEF SUMMARY OF THE INVENTION

The present invention provides for the efficient and economical production of efficacious super polyvalent vaccines against numerous types of pathogens. This is accomplished by taking advantage of the properties of nanodisks (alternatively spelled “nanodiscs”). Nanodisks are disk-shaped structures comprised of a loop composed of two molecules of an amphipathic protein, the MSP. This loop is spanned by a lipid membrane. These structures range from several to tens of nanometers across. They are easily produced through a self-assembly process. Polypeptide immunogens may be easily associated with, or incorporated in, the lipid membrane during the self-assembly process. Since the lipid membrane presents an electronic environment similar to the native environment of the immunogen (i.e., when it is part of the pathogen), the immunogen assumes the same, or nearly the same, conformation as when part of the pathogen.

This allows the adaptive immune system, when exposed to nanodisk-associated immunogens, to create antibodies which will bind to the immunogens when they appear on the target pathogens. Since minor chemical and structural differences in the immunogen will usually still allow the antibody to bind, even though the antibodies may display a lesser binding affinity for these epitopes, it is not necessary to have representatives of every possible immunogen epitope present in a vaccine formulation in order to have a vaccine which would be effective against all possible strains, or at least the infective strains, of the target pathogen or pathogens. Because of the cross-reactivity of antibodies with epitopes of the specific immunogen that they formed against, as long as they are chemically and/or structurally similar, it is not only unnecessary to have representatives of every possible epitope, it is also unnecessary to utilize the same dosage, for each epitope present, that would be required if a single epitope (“monovalent”) was being utilized. This is because of the summation of the binding affinities of the antibody epitopes which are formed against the immunogen epitopes presented combined with the cross-reactivity for the similar immunogen epitopes, presented or not.

Recombinant DNA technology is used to produce the MSP needed to create the nanodisks. This technology is also utilized to create and express, in bulk, the various immunogen epitopes required. Computer modeling of the immunogen may be used to determine which epitopes need to be created. These polypeptides are then created using standard recombinant DNA (“rDNA”) techniques and associated with, or incorporated in, the nanodisks during the self-assembly process. The nanodisks may then be purified through various means, including, but not limited to, size-exclusion chromatography (“SEC”).

These nanodisks are water soluble and may be formulated as vaccines. The MSPs may be produced so that they have chemical “tags” which allow for their attachment to appropriate substrates. Such “tagged” nanodisks, with the appropriate immunogen epitopes, attached to appropriate support or supports would be useful for isolating, extracting, or purifying chemical species, such as antibodies, or biological species, such as viruses, bacteria, or other microbes, where the capabilities of the present invention, such as the ability to bind a spectrum of epitopes or strains of the species, may be desirable. Furthermore, with an appropriate tag, such as described previously or with a fluorophore or similar tag, the nanodisk collections following under the present invention, even if untagged, may find use in assays, screening tests, or other tests which would advantageously utilize their spectrum of binding affinities.

### BRIEF DESCRIPTION OF THE SEVERAL VIEWS OF THE DRAWING

The foregoing aspects and others will be readily appreciated by the skilled artisan from the following description of illustrative embodiments when read in conjunction with the accompanying drawing.

The drawing illustrates a nanodisk in which a polypeptide immunogen, here trimeric rHA, is associated with the phospholipid bilayer of the nanodisk.

### DETAILED DESCRIPTION OF THE INVENTION

Before the invention is described in detail, it is to be understood that, unless otherwise indicated, this invention is not limited to particular embodiments, materials, and processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly indicates otherwise.

In this specification and the appended claims, reference will be made to a number of terms that shall be defined to have the following meanings:

The terms “optional” or “optionally” mean that the subsequently described feature or structure may or may not be present, or that the subsequently described event or circumstances may or may not occur, and that the description includes instances where it is not, or instances where the event or circumstances occurs and instances where it does not.

The term “nanodisk” means a hydrophobic membrane composed of lipids or phospholipids which is surrounded by two amphipathic protein molecules which render the resulting disk-shaped structure water-soluble. The “nanodisk” has a diameter of several nanometers to tens of nanometers. An alternative spelling of “nanodisk” is “nanodisc.”

The term “polypeptide” means a molecule consisting of one or more chains of amino acid residues, or a protein.

The term “immunogen” means a molecule, often a polypeptide, which, when presented to the immune system of a healthy animal to which said molecule is foreign, will elicit some form of immune response against said molecule or a cell, or other organism, displaying said molecule.

The term “antigen” means an immunogen in which, at least part of, the immune response consists of the production of antibodies against said immunogen.

The term “epitope” means, depending on context, a variation or analog of a particular immunogen or antibody.

The term “conformation” means, depending upon context, the three-dimensional structure of a molecule or the electronic structure displayed at the surface of said molecule which determines the binding affinity between said molecule and another molecule.

The term “target pathogen” means the biological species, or other division of biological entity, which presents the epitope or epitopes of immunogen or immunogens which are, depending upon context, associated with the membranes of the nanodisks in a particular embodiment or formulation or which would be similarly affected by the immune response to said epitope or epitopes, unless context indicates otherwise.

The term “collection” means, unless context indicates otherwise, a set or group of the item or items denoted; wherein said set or group is comprised of two or more of said item or items; wherein said set or group is comprised of two or more variations of at least one said item; wherein said variation consists of an alteration to, or variation in, at least one functional element of said item; and wherein said alteration, or variation, produces a difference in the function, or a functional property, of the item from that of some other member of the set without said alteration, or variation.

The term “polyvalent” means that, depending on the context, the collection, vaccine, assay, test, or other embodiment is efficacious against, or binds to, two or more strains, species, epitopes, or other group, of pathogen, antibody, other biological entity, or other chemical species.

The term “super polyvalent” means that, depending on the context, a polyvalent collection, vaccine, assay, test, or other embodiment is efficacious against or binds to, five or more, or all possible, either infective or not, strains, species, epitopes, or other group, of pathogen, antibody, other biological entity, or other chemical species.

The terms “associated with,” “nanodisk-associated,” and “membrane-associated” mean that the molecule is bound to, incorporated in, or chemically tethered to, the membrane of a nanodisk, unless context indicates otherwise.

Before embarking on descriptions of particular embodiments of the present invention, it would be beneficial to review some of the relevant theory.

When an animal's adaptive immune system encounters certain molecules, which, normally, are foreign to the animal, it will produce molecules, called antibodies, which will bind with these molecules. These molecules which trigger antibody production are called “antigens.” This is the process by which humoral immunity is conferred. If the antigen is presented on a pathogen, such as a virus, after the animal has produced antibodies to it, the antibodies to that antigen will bind to the antigen molecules. This may block the antigen from binding to particular sites on the cells of the animal and thus inhibit infection by the pathogen. The bound antibody may also act as a chemical marker, or act as the attachment point for another chemical marker, which indentifies, or primes, the pathogen for destruction by other components of the animal's immune system.

The goal of vaccination is to produce long-term resistance to infection by the target pathogen. This immunity is often determined by the levels of a class of antibody, IgG, capable of binding with the specific antigen, which is present in the blood of the vaccinated subject. IgG is the antibody class chosen for the test since it is produced indefinitely subsequent to initial antigen presentation.

Since the functionality of a particular antibody is dependent upon its ability to bind to specific antigens, the structure, especially the electronic structure displayed at the surface of the physical structure (“conformation”) of the antigen is important to the production of functional antibodies. Thus, during vaccination, it is desirable for the target pathogen's antigen to be presented to the adaptive immune system in the proper conformation.

Outside of the body's immune system, the antigen specificity of an antibody's binding affinity has been advantageously utilized in various qualitative and quantitative tests. These tests primarily consist of presence detection and qualitative assays.

This binding specificity has also been utilized to isolate, extract, or purify the antigen, the antibody, or some other object bearing either of the molecules or binding to them.

Antibody production is not the only means by which the animal's immune system may defend against an infection. A cell-mediated response may also be triggered in which “effector” and “memory” T cells are produced. The T cells, of which the memory T cells confer long-term immunity to the animal, are keyed to a segment, or segments, of a polypeptide immunogen.

Many immunogens, such as polypeptides, are dependent upon their electronic environment for their conformation. In other words, they assume different conformations when in different electronic environments. Different electronic environments may be presented by different solvent systems. For example, water presents a very different electronic environment to the immunogen than does a lipid or phospholipid membrane.

Immunogens are often presented, by a potential pathogen, associated with a lipid or phospholipid membrane. This constitutes their “native” environment. In this “native” environment they assume their “native” conformation.

Many pathogens evade the immune system, and create vaccination difficulties, by frequently altering (mutating) the amino acid sequence of their polypeptide immunogens or by differentiating into numerous strains, each of which displays minor alterations (or mutations) in the immunogens they present. An ideal effective vaccine against such a pathogen would be effective against all such strains of the pathogen which may be infective. In other words, it would elicit the production of a collection of antibodies which would effectively bind all mutations of the pathogen's immunogen which would still permit infection to occur.

The present invention utilizes recombinant DNA technology to create a collection of mutated immunogens which are then associated with the lipid, or phospholipid, membranes of nanodisks. The nanodisk membrane provides an environment suitable for the immunogen to assume its native conformation.

Furthermore, the nanodisk effectively renders the immunogen, and membrane, water-soluble. This is advantageous for many applications including, but not limited to, vaccine formulation.

The nanodisk, especially the MSP, may also incorporate functionality beyond hosting the immunogen. Some examples of this functionality include attachment of chemical “tags” for anchoring the nanodisk to certain substrates or supports. Incorporation of a fluorophore which would activate upon antibody binding may also be accomplished and applied advantageously.

The selection of which nanodisk-associated immunogen epitopes to be formulated into any particular collection, or embodiment, depends upon the purpose of said collection, or embodiment. The present invention allows for many possibilities.

Antigen/antibody cross-reactivity (the ability of an antibody to still bind to an antigen which is not specifically the one it was formed against) enables a collection of nanodisk-associated immunogens to bind a larger number of antibody epitopes than the number of immunogen epitopes contained in said collection. Likewise, the antibodies produced by the immune system in response to exposure to a collection of nanodisk-associated immunogen epitopes are capable, as a group, of effectively binding a larger number of immunogen epitopes than was presented in the collection of nanodisk-associated immunogen epitopes. Therefore, to achieve immunity against an entire genus or species of pathogen, it is not necessary to produce a collection of nanodisk-associated immunogen epitopes wherein every possible epitope is represented. Computer modeling may be utilized to select a spectrum of epitopes whose conformations are complementary and will elicit a spectrum of antibodies that will be efficacious in binding to the full range of immunogen epitopes, or at least those which would be displayed by strains of the pathogen capable of producing infection.

Now turning to the preferred embodiments of the present invention. The drawing illustrates a nanodisk 100 with a membrane-associated immunogen 200, which in this instance is the trimer of the influenza A antigen, hemagglutinin (“HA”), produced via recombinant DNA techniques. This nanodisk would be a constituent of a vaccine against influenza A.

To produce said vaccine, the epitopes of influenza A HA required to provide immunity against the desired spectrum of strains of influenza A are selected. These epitopes are then produced through conventional recombinant DNA techniques, isolated, and purified.

The purified recombinant-origin HA (“rHA”) epitopes are then used during nanodisk assembly to create the nanodisk-associated rHA. This is done in single epitope batches since HA will form multimers, such as the trimer 200 illustrated. By assembling the nanodisks in single epitope batches, one is assured that the rHA molecules 210, 220, and 230 in the trimer 200 are all of the same epitope as they would be in a natural pathogen.

Nanodisk self-assembly is accomplished as described by Denisov et al and Bhattacharya et al. Briefly, an appropriate MSP 150, which may also be produced utilizing recombinant DNA techniques, is combined with a solution of the phospholipid 111, palmitoyloleoyl phosphatidylcholine (“POPC”) in this instance, dissolved in a Tris-HCl and NaCl buffer containing sodium cholate solution and incubated at 0° C. The rHA epitope is solubilized in beta-octylglucoside and added to the MSP-POPC-cholate mixture and incubated at 0° C. Self-assembly may be initiated by the addition of SM-2 Bio-Beads (from Bio-Rad, Hercules, Calif., USA) and incubation continued at 0° C. while keeping the beads suspended. The nanodisks containing rHA may be purified through immobilized metal affinity chromatography (“IMAC”).

Centrifugal ultrafiltration may be utilized to concentrate the IMAC eluates and size-exclusion chromatography (“SEC”) applied to fractionate the various nanodisk-associated immunogens, into, for example, empty nanodisks, those with a rHA monomer, those with a rHA dimer, those with a rHA trimer, and those with higher rHA oligomers. This application of SEC is optional as the empty nanodisk described here do not seem to be toxic.

When the rHA epitopes have been nanodisk-associated, an intranasally-delivered vaccine may be prepared by placing the appropriate portions of the nanodisk-associated rHA epitopes in an appropriate buffer (Tris-saline for example) to create the required dosing concentrations. Because of cross-reactivity, the dosage of any particular immunogen epitope in the collection will be less than it would be if used alone.

It is not necessary to formulate the vaccine so that all the required immunogen epitopes are represented in a single solution or mixture. The vaccine may instead be formulated as a series of solutions, or mixtures, each of which contains some portion of the total number of the required nanodisk-associated immunogen epitopes. These may be administered as a series over time until the subject has received the full complement of nanodisk-associated immunogen epitopes necessary.

Such an intranasal super polyvalent vaccine could be formulated for use against all influenza A strains. This would remove the need to take a polyvalent vaccine representing only three strains usually every year. Because of how it is produced, it is devoid of the presence of chicken egg proteins and may thus be used by individuals who are hypersensitive to such proteins. Another advantage over current commercially available vaccines is that it may very quickly be designed and produced, relatively cheaply, in bulk.

HIV is another virus whose infectivity benefits from the high mutation rate of the immunogens that it displays. Therefore, a HIV vaccine may be prepared by following the procedures described herein, but utilizing a HIV immunogen, such as gp120, in place of rHA. It may be beneficial to utilize a mixture of HIV immunogens, such as gp120 and gp41, if said immunogens are complexed together in the pathogen.

Whereas the influenza A vaccine described herein is effective at eliciting an efficacious humoral immune response whether administered intranasally or intramuscularly, for some pathogens, such as HIV, intranasal delivery may not produce an effective immune response.

The nanodisk-associated immunogen super polyvalent vaccines described herein may also be formulated to be efficacious against numerous species, genuses, or types of pathogens. This includes producing vaccine formulations that are effective against mixtures of pathogens. The present invention also allows for the super polyvalent collections of nanodisk-associated immunogens to be utilized for other purposes including assays, detection and other tests, isolation, extraction, and purification of various biological entities and chemical species. These super polyvalent nanodisk-associated immunogens may be developed and produced quickly, efficiently, and relatively cheaply in comparison with conventional in vivo production techniques done in animal cell systems. This also makes the present invention suitable for dealing with emergent biological threats.

### REFERENCES

P. Bhattacharya et al, Nanodisc-Incorporated Hemagglutinin Provides Protective Immunity against Influenza Virus Infection, Journal of Virology 84 (1): 361-371 (2010).
W. Fiers, S. Neirynck, T. Deroo, i. Saelens, and W. M. Jou, Soluble recombinant influenza vaccines, Philos. Trans. R. Soc. London B Biol. Sci. 356: 1961-1963 (2001).
S. P. McBurney, K. R. Young, and T. M. Ross, Membrane embedded HIV-1 envelope on the surface of a virus-like particle elicits broader immune response than soluble envelopes, Virology 358; 334-346 (2007).
T. H. Bayburt, J. W. Carlson, and S. G. Sligar, Reconstitution and imaging of a membrane protein in a nanometer-size phospholipid bilayer, J. Struct. Biol. 123: 37-44 (1998).
K. J. Metzner, Detection and significance of minority quasispecies of drug-resistant HIV-1, J. HIV Ther. 11 (4): 74-81 (2006).
M. R. Sandbulte et al, Discordant antigenic drift of neuraminidase and hemagglutinin in H1N1 and H3N2 influenza viruses, Proc. Nat. Acad. Sci. USA 108 (51): 20748-20753 (2011).
J. Borch, F. Torta, S. G. Sligar, and P. Roepstorff, Nanodiscs for Immobilization of Lipid Bilayers and Membrane Receptors: Kigetic Analysis of Cholera Toxin Binding to a Glycolipid Receptor, Analytical Chemistry 80 (16): 6245-6252 (2008).
I. G. Denisov, Y. V. Ginkova, A. A. Lazarides, and S. G. Sligar, Directed self-assembly of monodisperse phospholipid bilayer Nanodiscs with controlled size, J. Am. Chem. Soc 126: 3471-3487 (2004).

We claim:

1) A composition of matter comprising:
(a) a collection of nanodisks;
(b) where within said collection, one or more polypeptide immunogens are associated with, bound to, embedded within, or tethered to, the membranes of the individual nanodisks; and
(c) wherein said collection contains five or more epitopes of at least one of said immunogens.

2) The composition of claim 1 when used as a component of an assay, detection kit, or other test.

3) The composition of claim 1 when used to isolate, extract, or purify, a chemical species, or biological entity, which selectively binds to said immunogen.

4) The composition of claim 1 when used as a component of a vaccine.

5) The composition of claim 4 when used as a component of a vaccine against strains of influenza A virus.

6) The composition of claim 4 when used as a component of a vaccine against strains of human immunodeficency virus.
